(19) United States
(12) Patent Application Publication
(10) Pub. No.: US 2022/0228108 A1
(43) Pub. Date: Jul. 21, 2022

(54) CELL CULTURE BASE MATERIAL AND CELL CULTURE BASE MATERIAL WITH CELLS

(71) Applicant: FUJIFILM CORPORATION, Tokyo (JP)

(72) Inventors: Keisuke OKU, Kanagawa (JP); Daichi HIKIMOTO, Kanagawa (JP); Takahiro OBA, Kanagawa (JP); Kenichi YASUDA, Kanagawa (JP); Hiroyuki YUKAWA, Kanagawa (JP)

(21) Appl. No.: 17/658,446

(22) Filed: Apr. 8, 2022

Related U.S. Application Data
(63) Continuation of application No. PCT/JP2020/039713, filed on Oct. 22, 2020.

(30) Foreign Application Priority Data
Oct. 25, 2019 (JP) 2019-194541

(51) Int. Cl. C12N 5/00

(52) U.S. Cl. CPC C12N 5/0068; C12N 2533/30; C12N 2533/54

(57) ABSTRACT

There is provided a cell culture base material having a porous membrane having an opening ratio of 30% to 70% and an extracellular matrix with which an inside of a hole of the porous membrane is filled. There is also provided a cell culture base material with cells having a cell layer on at least one surface of the cell culture base material.

### CROSS-REFERENCE TO RELATED APPLICATIONS

This application is a continuation application of International Application No. PCT/JP2020/039713, filed Oct. 22, 2020, the disclosure of which is incorporated herein by reference in its entirety. Further, this application claims priority from Japanese Patent Application No. 2019-194541, filed Oct. 25, 2019, the disclosure of which is incorporated herein by reference in its entirety.

### BACKGROUND OF THE INVENTION

### 1. Field of the Invention

The present disclosure relates to a cell culture base material and a cell culture base material with cells.

### 2. Description of the Related Art

Cell culture technique has attracted attention as a tool for not only regenerative medicine but also drug discovery support. In the related art, although a cell culture base material that serves as a scaffold for cell culture has been mainly used in planar culture, various improvements have been attempted depending on the intended purpose such as improvement of biomimeticity.

As a culture base material for easily and accurately evaluating the infiltration ability of cells, JP2002-320472A proposes a coated membrane obtained by coating a porous membrane, such as a track-etched polyethylene terephthalate (PET) membrane, with a composition containing a reconstituted and aggregated extracellular matrix.

JP2006-500953A proposes a cell culture scaffold material in which biologically active molecules such as an extracellular matrix molecule, a growth factor, and a signal transduction molecule are incorporated in a porous hydrogel by non-covalent bonding.

As a porous body having excellent biocompatibility and mechanical strength, JP2017-52829A proposes a coated porous body obtained by coating a porous body with a composition containing silk fibroin and alcohol. Further, it is disclosed that the above-described coated porous body can be applied to a cell culture support and the like.

For the purpose of producing a cell laminate, WO2018/225835A proposes a method of culturing cells on both surfaces of a porous membrane to form a cell layer on both surfaces of the porous membrane.

### SUMMARY OF THE INVENTION

In recent years, it has been known that in a case where a mechanical stimulus is applied to cells during cell culture, biomimeticity and the like can be improved. Further, in the evaluation of drug toxicity or the like, it may be useful to carry out the evaluation while applying a mechanical stimulus, which mimics that of a living body, to the cultured cells. In a case where a cell culture base material that serves as a scaffold can be deformed, it is possible to apply a mechanical stimulus such as extension tension to cells due to the above deformation. For this reason, the inventors attempted to develop a cell culture base material suitable for deformation.

In the planar culture technique in the related art, a cell culture base material suitable for deformation is not known. Further, even in the cell culture base material in the related art, in which a porous membrane is used, a cell culture base material suitable for deformation and having good cell adhesiveness has not been obtained. For example, as described in JP2002-320472A, a track-etched PET membrane is widely used as a porous membrane for cell culture; however, the track-etched PET membrane generally has a low opening ratio of, for example, about 2% to 20%, and thus it is hard to be deformed. In a case where a porous membrane having a higher opening ratio is used, a cell culture base material more suitable for deformation can be obtained; however, since the contact area between cells and the cell culture base material becomes small, it is difficult to secure cell adhesiveness in the case of the porous membrane having a higher opening ratio.

In consideration of the above circumstances, an object of one embodiment of the present disclosure is to provide a cell culture base material which is easily deformable and has good cell adhesiveness, and a cell culture base material with cells, which is easily deformable and in which cells well adhere to the base material.

The means for achieving the above-described object include the following aspects.

<1> A cell culture base material comprising

a porous membrane having an opening ratio of 30% to 70%; and

an extracellular matrix with which an inside of a hole of the porous membrane is filled.

<2> The cell culture base material according to <1>, in which the porous membrane has an average opening diameter of 1 μm to 200 μm.

<3> The cell culture base material according to <1> or <2>, in which the cell culture base material has a thickness of 20 μm or less.

<4> The cell culture base material according to any one of <1> to <3>, in which a filling rate of the hole with the extracellular matrix is 80% or more.

<5> The cell culture base material according to any one of <1> to <4>, in which the extracellular matrix has a gel shape or is capable of forming a gel in a moist environment.

<6> The cell culture base material according to any one of <1> to <5>, in which a Young's modulus, determined by a tensile test based on JIS K 7161-1: 2014 and JIS K 7127: 1999, is 2.0 MPa or less.

<7> The cell culture base material according to any one of <1> to <6>, in which a maximum elongation rate, determined by a tensile test based on JIS K 7161-1: 2014 and JIS K 7127: 1999, is 150% or more.

<8> The cell culture base material according to any one of <1> to <7>, in which at least one surface of the porous membrane is coated with an extracellular matrix.

<9> A cell culture base material with cells, comprising a cell layer on at least one surface of the cell culture base material according to any one of <1> to <8>.

According to one embodiment of the present disclosure, there are provided a cell culture base material which is easily deformable and has good cell adhesiveness, and a cell culture base material with cells, which is easily deformable and in which cells well adhere to the base material.

### BRIEF DESCRIPTION OF THE DRAWINGS

FIG. 1A is a perspective view illustrating an example of a porous membrane having a honeycomb structure.

FIG. 1B is a plan view of the porous membrane in FIG. 1A in a case of being viewed from the upper surface side thereof.

FIG. 1C is a cross-sectional view taken along a line C-C of the porous membrane in FIG. 1B.

FIG. 2 is scanning electron microscope (SEM) images of a honeycomb film used in the production of a cell culture base material in Example 1.

FIG. 3 is scanning electron microscope (SEM) images of a cell culture base material produced in Example 1.

FIG. 4 is microscopic images of a base material A (the left figure) and a base material C (the right figure), produced in Example 2.

FIG. 5 is microscopic images of cells stained with VE-cadherin, which are cultured in Example 2.

FIG. 6 is a graph showing the Young's modulus and the maximum elongation rate of a base material used in Example 3.

FIG. 7 is a table showing the Young's modulus and the maximum elongation rate of the base material used in Example 3.

### DESCRIPTION OF THE PREFERRED EMBODIMENTS

Hereinafter, embodiments of the present disclosure will be described. These descriptions and Examples are only illustrative of the embodiments and do not limit the scope of the invention.

The numerical range indicated by using “to” in the present disclosure indicates a range including numerical values described before and after “to” as a lower limit value and an upper limit value, respectively.

The term “step” in the present disclosure not only includes an independent step, but also includes a step that may not be clearly distinguished from the other step but still achieves a desired effect of the step.

In the present disclosure, upon referring to an amount of each component in a composition, the amount means a total amount of a plurality of substances present in the composition unless otherwise specified, in a case where a plurality of substances corresponding to each component are present in the composition.

In the present disclosure, a combination of two or more preferred aspects is a more preferred aspect.

In the present disclosure, the coefficient of variation is expressed in terms of percentage. The coefficient of variation is a value obtained by dividing a standard deviation by a mean value for a certain population and is an indicator showing the degree of variability of the population.

In a case where an embodiment is described in the present disclosure with reference to the accompanying drawings, the configuration of the corresponding embodiment is not limited to the configuration shown in the drawings. In addition, the size of the member in each drawing is conceptual, and the relative relationship between the sizes of the members is not limited thereto. In addition, members having substantially the same function in each drawing are given the same reference numerals in all drawings, and any redundant description may be omitted.

«Cell Culture Base Material»

A cell culture base material of the present disclosure has a porous membrane having an opening ratio of 30% to 70% and an extracellular matrix with which an inside of a hole of the porous membrane is filled. The cell culture base material of the present disclosure has a porous membrane having an opening ratio of 30% or more, and thus even in a case where stress for deformation is applied, it is hard to be broken and has excellent deformability as compared with a case where a membrane having a lower opening ratio is included. On the other hand, since the cell culture base material of the present disclosure has a porous membrane having a relatively high opening ratio, such as an opening ratio of 30% or more, while the inside of the hole of the porous membrane are filled with an extracellular matrix, a large cell adhesiveness area can be secured, whereby cell adhesiveness is excellent. Further, according to the cell culture base material of the present disclosure, for example, the trouble that cells fall off to the back side of the holes is reduced during cell culture or when the cell culture base material is deformed in order to apply a mechanical stimulus such as extension tension, and thus it is possible maintain good cell adhesiveness.

Further, according to the cell culture base material of the present disclosure, since the porous membrane has an opening ratio of 70% or less, the cell culture base material of the present disclosure can exhibit a self-supporting property while having such excellent deformability as described above.

In addition, in a case where cell culture is carried out using a porous membrane having a high opening ratio in the related art, the contact area with respect to the cell scaffold is small, and thus the morphology, function, and the like of the cultured cells may be different from those in the case of planar culture. On the other hand, the cell culture base material of the present disclosure is also useful in that it enables cell culture under conditions close to those of planar culture.

Hereinafter, the porous membrane and the extracellular matrix will be described in detail.

<Porous Membrane>

The porous membrane that is used in the cell culture base material of the present disclosure functions as a scaffold to which cells adhere. The kind of porous membrane is not particularly limited as long as the porous membrane is a porous membrane having an opening ratio of 30% to 70%. In the present disclosure, the “holes” of the porous membrane mean spaces present in the membrane, which are partitioned from each other by partition walls. However, adjacent holes may partially communicate with each other.

The material of the porous membrane is not particularly limited. Examples of the material of the porous membrane include polymers such as polybutadiene, polystyrene, polycarbonate, polyester (for example, polylactic acid, polycaprolactone, polyglycolic acid, a polylactic acid-polyglycolic acid copolymer, a polylactic acid-polycaprolactone copolymer, polyethylene terephthalate, polyethylene naphthalate, polyethylene succinate, polybutylene succinate, and poly-3-hydroxybutyrate), polyacrylate, polymethacrylate, polyacrylamide, polymethacrylamide, polyvinyl chloride, polyvinylidene chloride, polyvinylidene fluoride, polyhexafluoropropene, polyvinyl ether, polyvinylcarbazole, polyvinyl acetate, polytetrafluoroethylene, polylactone, polyamide, polyimide, polyurethane, polyurea, polyaromatics, polysulfone, polyethersulfone, a polysiloxane derivative, and a cellulose acylate (for example, triacetyl cellulose, cellulose acetate propionate, cellulose acetate butyrate).

The polymer may be a homopolymer, a copolymer, a polymer blend, or a polymer alloy, as necessary, from the viewpoints of solubility in a solvent, optical properties, electrical properties, membrane strength, elasticity, and the like. One kind of polymer may be used singly or two or more kinds thereof may be used in combination.

The material of the porous membrane is preferably at least one polymer selected from the group consisting of polybutadiene, polyurethane, polystyrene, and polycarbonate, from the viewpoint of the self-supporting property. From the viewpoint of easily maintaining the engraftment of the cell layer, it is preferably at least one polymer selected from the group consisting of polylactic acid, a polylactic acid-polyglycolic acid copolymer, and a polylactic acid-polycaprolactone copolymer. From the viewpoint of achieving better deformability, it is preferably an elastomer of polybutadiene, polyurethane, or the like.

An example of the porous membrane will be described below with reference to the drawings. In the following description, the “major axis” means a maximum length among distances between any two points on the contour; however, in a case where the direction is specified, it means a maximum length among distances between any two points in that direction.

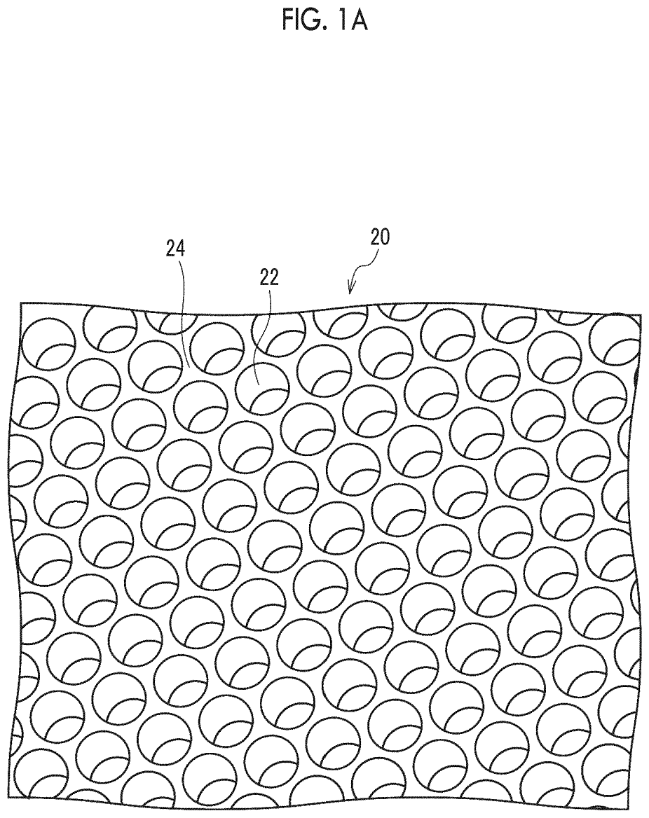

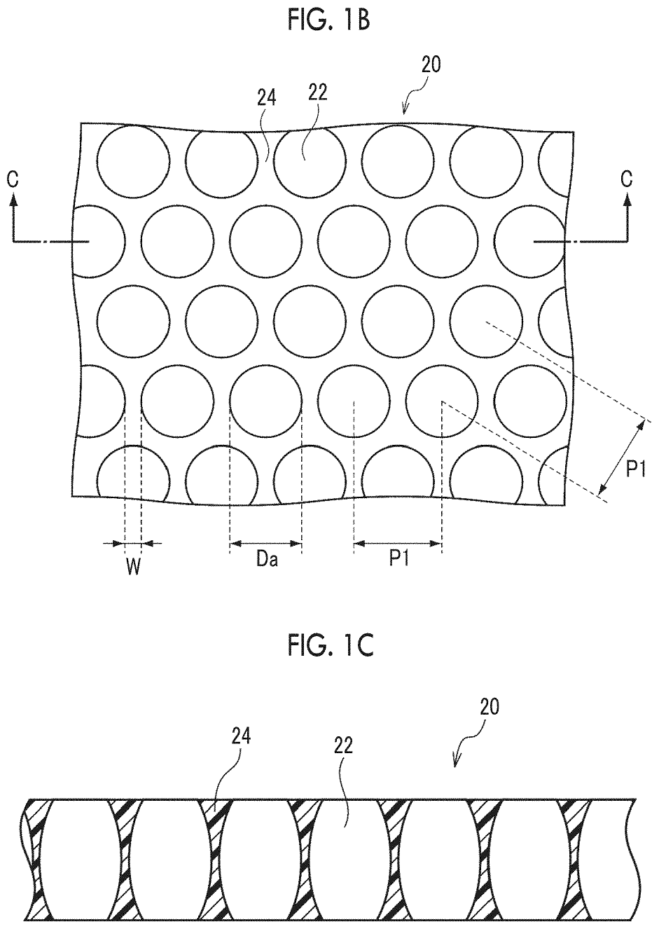

FIG. 1A to FIG. 1C are views illustrating a porous membrane 20 which is an example of the porous membrane. FIG. 1A is a perspective view of the porous membrane 20, FIG. 1B is a plan view of the porous membrane 20 in FIG. 1A in a case of being viewed from the upper surface side thereof, and FIG. 1C is a cross-sectional view of the porous membrane 20 taken along a line C-C in FIG. 1B.

Holes 22 are arranged over the entire main surface of the porous membrane 20. However, in a case where the porous membrane 20 has a region with which cells cannot come into contact, the holes 22 may not be arranged in the region with which the cells cannot come into contact. In the porous membrane 20, adjacent holes 22 are separated from each other by a partition wall 24.

Although adjacent holes 22 are not communicated with each other in FIG. 1A to FIG. 1C, the adjacent holes 22 may be partially communicated with each other by a communication hole. Even in a case where the adjacent holes 22 are partially communicated with each other by the communication hole, they are regarded as separate holes partitioned by the partition wall 24.

In FIG. 1A to FIG. 1C, the hole 22 is a through-hole, but the hole 22 may be a non-through-hole. In a case of carrying out both faced culture, in which cells of the same origin or cells of the heterologous origin are cultured on each of both surfaces of the porous membrane, the hole of the porous membrane is preferably a through-hole from the viewpoint of promoting intercellular interaction on both surfaces of the porous membrane. Further, from the viewpoint of further improving the deformability, the hole 22 is preferably a through-hole.

The porous membrane 20 illustrated in FIG. 1A to FIG. 1C has a honeycomb structure. The honeycomb structure means a structure in which holes are arranged in a honeycomb shape. The honeycomb-shaped arrangement refers to an arrangement in which a parallel hexagon (preferably a regular hexagon) or a shape close thereto is used as a unit and the centroid of the opening is located at the apex and the point of intersection of diagonal lines of such a geometrical figure. The “centroid of opening” means the centroid of the opening of a two-dimensional geometrical figure on the main surface. Since the porous membrane 20 has a honeycomb structure, it is possible to increase the opening ratio, and thus it is possible to obtain better deformability. Further, in a case where cells are subjected to both faced culture using the porous membrane 20, it is preferable to increase the opening ratio also due to the reason that the intercellular interaction on each surface is efficiently carried out.

The arrangement of the inside of the hole of the porous membrane 20 is not limited to the honeycomb structure, and the porous membrane 20 may have a lattice form arrangement, a face-centered lattice form arrangement, or the like.

The lattice form arrangement refers to an arrangement in which a parallelogram (needless to say, a square, a rectangle, and a rhombus shape is included therein, where a square is preferable) or a shape close thereto is used as a unit, and the centroid of the opening is located at the apex of such a geometrical figure.

The face-centered lattice form arrangement refers to an arrangement in which a parallelogram (needless to say, a square, a rectangle, and a rhombus shape is included therein, where a square is preferable) or a shape close thereto is used as a unit, and the centroid of the opening is located at the apex and the point of intersection of diagonal lines of such a geometrical figure.

From the viewpoint of enhancing the homogeneity of the cell layer that is formed on the porous membrane, it is preferable that the holes 22 in the porous membrane 20 are regularly arranged. As a reference guideline for being regularly arranged, there is an arrangement in which, regarding the area of the parallel hexagonal shape or the parallelogram shape which is the unit of arrangement, the coefficient of variation thereof is 10% or less. The coefficient of variation is obtained for any 10 units of arrangement.

The shape of the hole 22 is not particularly limited. Examples of the shape of the hole 22 include a truncated spherical shape obtained by cutting out a part of a sphere, a barrel shape, a cylindrical shape, and a prismatic shape.

Examples of the shape of the opening of the hole 22 include a circular shape, an elliptical shape, and a polygonal shape. The opening of the porous membrane 20 means an inlet portion of the hole 22 formed in at least one of the two main surfaces of the porous membrane 20.

Hereinafter, the dimensions of the porous membrane 20 will be described.

The opening ratio of the porous membrane 20 is 30% to 70%. Since the opening ratio of the porous membrane is 30% or more, it is possible to produce a cell culture base material having excellent deformability. Moreover, since the opening ratio of the porous membrane is 70% or less, the self-supporting property is excellent. From this viewpoint, the opening ratio of the porous membrane is preferably 30% to 60% and more preferably 35% to 50%.

In the present disclosure, the opening ratio of the porous membrane refers to a proportion of the total area of the opening to the total area of the cell culture region (including the area of the opening) in the plan view of the opening surface of the porous membrane (that is, the surface having the opening of the porous membrane). The cell culture region means a region with which cells can come into contact by seeding. A region of the porous membrane 20 on the opening surface thereof, with which cells cannot come into contact, is not included in the cell culture region. In a case where openings are present on both surfaces of the porous membrane, the opening ratio on at least one surface is 30% to 70%.

The pitch P1 of the holes 22 is the distance between the centers of the adjacent openings. The pitch P1 is preferably set depending on the size of the cells to be cultured on the porous membrane 20. The pitch P1 may be, for example, 1 μm to 50 μm.

The opening diameter Da is defined as a major axis of the opening of the hole 22. The average opening diameter, which is the average value of the opening diameters Da, may be, for example, 10% to 150% with respect to the major axis (for example, 10 μm to 50 μm) of the cells to be seeded. The average opening diameter can be appropriately set depending on the intended purpose. From the viewpoint of good deformability, the average opening diameter is preferably 1 μm or more, more preferably 2 μm or more, and still more preferably 3 μm or more. From the viewpoint of the strength of the porous membrane 20, the average opening diameter is preferably 200 μm or less, more preferably 50 μm or less, and still more preferably 10 μm or less. From the above viewpoints, the average opening diameter is preferably 1 μm to 200 μm, more preferably 2 μm to 50 μm, and still more preferably 3 μm to 10 μm. The average opening diameter is determined as an arithmetic mean value of the opening diameters Da of any ten holes 22.

The coefficient of variation of the opening diameter Da is preferably 20% or less, and the smaller the coefficient of variation is, the more preferable it is. The smaller the coefficient of variation of the opening diameter Da is, the higher the homogeneity of the cell layer formed on the porous membrane 20 tends to be. The coefficient of variation of the opening diameter Da is determined for any 10 holes.

The width W of the partition wall 24 is the length of the width of the partition wall 24 on the line segment that connects the centers of the adjacent openings. From the viewpoints of maintaining the self-supporting property of the porous membrane and improving the handleability, the width W is preferably 0.5 μm or more, more preferably 1 μm or more, and still more preferably 3 μm or more.

From the viewpoint of producing a cell culture base material having a suitable thickness, the thickness of the porous membrane 20 is preferably 40 μm or less, more preferably 20 μm or less, still preferably 8 μm or less, particularly preferably 5 μm or less, and extremely preferably 3 μm or less. In addition, also similarly from the viewpoint of producing a cell culture base material having a suitable thickness, the thickness of the porous membrane 20 is preferably 0.5 μm or more, more preferably 1 μm or more, and still more preferably 1.5 μm or more. From the above viewpoints and from the viewpoints of being easily deformable and easily obtaining good cell adhesiveness, the thickness of the porous membrane 20 is preferably 0.5 μm to 40 μm, more preferably 1 μm to 20 μm, still more preferably 1.5 μm to 8 μm, particularly preferably 1.5 μm to 5 μm, and extremely preferably 1.5 μm to 3 μm.

The porous membrane 20 illustrated in FIG. 1A to FIG. 1C is a single-layer membrane; however, a laminated membrane obtained by laminating a plurality of porous membranes may be used for cell culture.

[Method of Manufacturing Porous Membrane]

A method of manufacturing a porous membrane is not particularly limited. Examples of the method of manufacturing a porous membrane include a method of subjecting a membrane made of a resin to etching processing, blasting processing, or punching processing, thereby forming through-holes to obtain a porous membrane; and manufacturing methods of causing water droplets to grow in a coating film containing a polymer and a solvent to form through-holes, which are disclosed in JP4734157B, JP4945281B, JP5405374B, JP5422230B, and JP2011-74140A.

<Extracellular Matrix>

In the cell culture base material of the present disclosure, the inside of the hole of the porous membrane is filled with an extracellular matrix. The extracellular matrix is biological macromolecules present outside the cell. In addition to functioning as a scaffold for cell culture, the extracellular matrix can also act on cell proliferation, differentiation, and phenotypic expression. Since the inside of the inside of the hole of the porous membrane is filled with the extracellular matrix, the cell adhesion surface can be widely secured, and a desired action due to the extracellular matrix can be suitably obtained.

Examples of the extracellular matrix include at least one kind of extracellular matrix selected from the group consisting of fibronectin, collagen (for example, type I collagen, type IV collagen, or type V collagen), laminin, vitronectin, gelatin, perlecan, nidogen, proteoglycan, osteopontin, tenascin, nephronectin, a basement membrane matrix, and polylysine. As the basement membrane matrix, a commercially available product (for example, MATRIGEL (registered trade name) or Geltrex (registered trade name)) is available.

In the present disclosure, the description that the inside of the hole of the porous membrane “is filled” with an extracellular matrix indicates that in a case where the hole is a through-hole, the extracellular matrix is retained in the hole to the extent that the through-hole becomes closed to be non-through-hole, and in a case where the holes is a non-through-hole, the extracellular matrix is retained in at least a part of the volume of the non-through-hole to fill the hole.

It is noted that the description that the inside of the hole of the porous membrane is “is filled” with an extracellular matrix does not necessarily mean that the entire volume of the holes inside the porous membrane is filled an extracellular matrix.

In addition, the extracellular matrix in the inside of the hole of the porous membrane may be in a moist state or a dry state. The description that the inside of the hole of the porous membrane “is filled” with an extracellular matrix means that in a case where an extracellular matrix is placed in a moist state, the inside of the hole of the porous membrane is in a state “being filled” with the extracellular matrix, the state being defined as described above. As a result, for example, even in a case where an extracellular matrix is in a dry state, it can be said that the inside of the hole of the porous membrane “is filled” with an extracellular matrix in a case where the through-hole becomes closed to be non-through-hole in a case where the extracellular matrix is in a moist state.

The extracellular matrix may be freeze-dried. In a case where the extracellular matrix is freeze-dried in a state where the inside of the hole of the porous membrane is filled with the extracellular matrix, the extracellular matrix tends to become in a dry state with the shape thereof being maintained in the hole.

In a case where a cell culture base material in a dry state is immersed in a liquid such as water or medium or placed in high humidity using an incubator or the like, it is possible to obtain a cell culture base material in which the inside of the hole of the porous membrane is filled with the extracellular matrix in the moist state.

Depending on the production operation of the cell culture base material, there may be a case where the extracellular matrix is not evenly disposed on the entire surface of the porous membrane, but is disposed on a part of the surface of the porous membrane and not disposed on the other part thereof, that is, a case of the unevenness in the disposition of the extracellular matrix. Even in such a case, it is understood by those skilled in the art that as long as the effect of the cell culture base material of the present disclosure is exhibited by the extracellular matrix which is partially disposed, it is within the scope of the cell culture base material of the present disclosure.

The filling rate of the hole by the extracellular matrix is preferably 60% or more, more preferably 80% or more, still more preferably 90% or more, and particularly preferably 100%.

In the present disclosure, the filling rate of the hole is measured as follows.

An extracellular matrix in a cell culture base material is stained by a method capable of staining the extracellular matrix. A cross-section observation is carried out on any cross section of the porous membrane using a microscope (magnification: 100 to 200 times). In the microscopic image, a proportion of the total area occupied by the extracellular matrix in the holes to the total area occupied by any 100 holes is defined as the filling rate of the hole.

In the present disclosure, the fact that the filling rate of the hole is 100% means that the entire hole is filled with an extracellular matrix in the visual field of observation.

It is noted that in a case where the cell culture base material is in a dry state (including a case of being freeze-dried), the filling rate is a value measured after the cell culture base material is made to be in a moist state.

Examples of the method capable of staining an extracellular matrix include staining with a picrosirius red staining kit.

In one embodiment of the present disclosure, the cell culture base material may be a base material in a state where at least one surface of the porous membrane is coated with an extracellular matrix, or may be a base material in a state where both surfaces of the porous membrane are coated with an extracellular matrix. From the viewpoint of further improving the adhesiveness to the cell culture base material, the cell culture base material is preferably a base material in a state where both surfaces of the porous membrane are coated with an extracellular matrix.

The description that the surface of the porous membrane “is coated with an extracellular matrix” refers to a state where the inside of the hole of the porous membrane is filled with an extracellular matrix and furthermore, the surface of the porous membrane is also coated with an extracellular matrix. In a case where at least one surface of the porous membrane is coated with an extracellular matrix, the adhesiveness (that is, cell adhesiveness) of the cells cultured on the above-described coated surface to the cell culture base material tends to be capable of being further improved.

In a case where at least one surface of the porous membrane is coated with an extracellular matrix, the thickness of the extracellular matrix, on the surface of the porous membrane, with which at least one surface of the porous membrane is coated is not particularly limited, and it may be, for example, a thickness of 0.01% to 30%, may be 0.01% to 20%, or may be 0.01% to 10%, with respect to the thickness of the porous membrane.

It is preferable that the extracellular matrix with which the inside of the hole of the porous membrane is filled has a gel shape or is in a state where a gel is capable of being formed in a moist environment. In a case where the gel-shaped extracellular matrix is used, the extracellular matrix can be well retained in the holes and the cell adhesiveness area can be secured well, and thus the cell adhesiveness is excellent.

In the present disclosure, “gel” or “having a gel shape” refers to a substance or a state, which is obtained by causing a colloidal dispersion system using a liquid as a dispersion medium to lose fluidity to be solidified, or a substance or a state, which has a three-dimensional network structure in which a polymer is crosslinked and which belongs to an intermediate between a solid and a liquid, which absorbs a solvent in the solvent and swells but is not dissolved.

In one preferred embodiment, the cell culture base material may include a porous membrane having through-holes, and a gel-shaped extracellular matrix which is retained in the inside of the hole of the porous membrane and with which the inside of the hole of the porous membrane is filled.

[Method of Producing Cell Culture Base Material]

A method of producing the cell culture base material is not particularly limited. For example, a cell culture base material may be produced by a production method in which the inside of the hole of the porous membrane is filled with a gel-shaped extracellular matrix, by (1) preparing a porous membrane having an opening ratio of 30% to 70%, (2) immersing the porous membrane in a solution containing an extracellular matrix, and (3) causing the extracellular matrix to be gelated.

In a case where the porous membrane is immersed in a solution containing an extracellular matrix, it is preferable that the porous membrane is immersed in the solution containing an extracellular matrix over the entire thickness of the porous membrane. By such a method, it is possible to suitably produce a cell culture base material having a planar surface. More preferably, the porous membrane is immersed in a solution containing an extracellular matrix so that the porous membrane is immersed in the solution containing an extracellular matrix over the entire thickness of the porous membrane and the amount of the solution containing an extracellular matrix is minimized. By such a method, it is possible to suitable produce a planar cell culture base material without excessively consuming the extracellular matrix, whereby the production cost tends to be reduced.

The concentration of the extracellular matrix solution can be adjusted appropriately. As an example, in a case where the extracellular matrix is collagen, the concentration of the collagen solution may be 0.3 mg/mL to 10 mg/mL, may be 1.0 mg/mL to 10 mg/mL, or may be 4.0 mg/mL to 10 mg/mL.

In a case of immersing the porous membrane in a solution containing an extracellular matrix, it is preferable to wash the porous membrane in advance with ethanol or the like. By such a method, it tends to be possible to suppress the remaining of voids between the porous membrane and the extracellular matrix.

A gelation method is not particularly limited, and examples thereof include heating and cooling, pH adjustment, and an addition of a crosslinking agent. For example, in a case where the extracellular matrix is collagen, the gelation may be carried out by carrying out a alkaline treatment using ammonia, a sodium hydroxide solution, or the like.

It is noted that instead of the step of immersing the porous membrane in the solution containing an extracellular matrix, the solution containing an extracellular matrix may be applied onto the porous membrane.

[Properties of Cell Culture Base Material]

(Thickness)

The thickness of the cell culture base material is preferably 40 μm or less, more preferably 20 μm or less, still preferably 8 μm or less, particularly preferably 5 μm or less, and extremely preferably 3 μm or less. In a case where the thickness is 40 μm or less, it is possible, for example, that cells on one surface and cells on the other surface interact well during both faced culture. From the viewpoint of the strength of the cell culture base material, the thickness of the cell culture base material is preferably 0.5 μm or more, more preferably 1 μm or more, and still more preferably 1.5 μm or more. From the above viewpoints and from the viewpoints of being easily deformable and easily obtaining good cell adhesiveness, the thickness of the cell culture base material is preferably 0.5 μm to 40 μm, more preferably 1 μm to 20 μm, still more preferably 1.5 μm to 8 μm, particularly preferably 1.5 μm to 5 μm, and extremely preferably 1.5 μm to 3 μm.

For example, a planar extracellular matrix membrane that does not use a porous membrane cannot maintain the self-supporting property and thus is inferior in handleability in a case where the thickness is reduced. However, in the cell culture base material of the present disclosure, the self-supporting property can be maintained even in a case where the thickness is, for example, 40 μm or less, preferably 20 μm or less, more preferably 8 μm or less, still more preferably 5 μm or less, and particularly preferably 3 μm or less, and thus it is useful in that both the deformability and the self-supporting property can be achieved even in a case where the thickness is reduced.

The thickness of the cell culture base material can be measured by microscopic observation.

(Young's Modulus)

The Young's modulus of the cell culture base material, which is determined by the tensile test based on JIS K 7161-1: 2014 and JIS K 7127: 1999, is preferably 2.0 MPa or less, more preferably 1.5 MPa or less, and still more preferably 1.2 MPa or less. A case where the above Young's modulus is 2.0 MPa or less indicates that the cell culture base material is excellent in deformability. The lower limit of the Young's modulus is not particularly limited, and the Young's modulus is preferably 0.1 MPa or more from the viewpoint of the strength of the cell culture base material.

From the viewpoint of maintaining the strength of the cell culture base material while excellent deformability is also obtained, the Young's modulus is preferably 0.1 MPa to 2.0 MPa, more preferably 0.1 MPa to 1.5 MPa, and still more preferably 0.1 MPa to 1.2 MPa.

Specifically, the Young's modulus can be determined by the method described in Examples.

(Maximum Elongation Rate)

The maximum elongation rate of the cell culture base material, which is determined by the tensile test based on JIS K 7161-1 and JIS K 7127: 1999, is preferably 130% or more, more preferably 140% or more, and still more preferably 150% or more. A case where the maximum elongation rate is 130% or more, preferably 140% or more, and more preferably 150% or more indicates that the cell culture base material is hard to be torn even in a case where it is elongated. The upper limit of the maximum elongation rate is not particularly limited, and the maximum elongation rate may be 500% or less from the viewpoint of handleability of the cell culture base material.

Specifically, the maximum elongation rate can be determined by the method described in Examples.

[Use Application of Cell Culture Base Material]

The use application of the cell culture base material is not particularly limited. The cell culture base material can be widely used in in vivo transplantation materials, tissue models for drug evaluation or pathological condition evaluation, test tissue preparation in place of the animal experiment, and the like. In particular, it can be suitably used in use applications where it is useful to apply a mechanical stimulus to cells during culture or evaluation. Further, according to the cell culture base material of the present disclosure, since the culture close to planar culture is possible, and it is possible to suppress such an event as cells pass through the holes of the porous membrane and fall off, it is suitable for preparing a tissue having a small number of defects such as opening.

The kind of cells to be cultured is not particularly limited. For example, the cell may be a dividing cell or a non-dividing cell. In the present disclosure, the “culture” does not necessarily have to be accompanied by cell proliferation and, this term includes a case where at least cell survival is maintained with or without proliferation.

Examples of the cells to be cultured include at least one kind of cell selected from the group consisting of a parenchymal cell (for example, a hepatic parenchymal cell or pancreatic parenchymal cell), a stromal cell (for example, a pericyte), a muscle cell (for example, a smooth muscle cell, a myocardial cell, or a skeletal muscle), a fibroblast, a nerve cell, an endothelial cells (for example, a vascular endothelial cell, or a lymphatic endothelial cell), an epithelial cell (for example, an alveolar epithelial cell, an oral epithelial cell, a bile duct epithelial cell, an intestinal epithelial cell, a pancreatic epithelial, a renal epithelial cell, a renal tubular epithelial cell, a placenta epithelial cells, and a cell (for example, a precursor cell, a mesenchymal stem cell, or a pluripotent stem cell) capable of differentiating into any one of these cells.

Examples of the pluripotent stem cell include an embryonic stem cell (an ES cell), an induced pluripotent stem cell (an iPS cell), an embryonic germ cell (an EG cell), an embryonal carcinoma cell (an EC cell), a multipotent adult progenitor cell (a MAP cell), an adult pluripotent stem cell (an APS cell), and a multi-lineage differentiating stress enduring cell (a Muse cell). A pluripotent stem cell can be differentiated into a somatic cell by adding, to a medium, a differentiation-inducing factor that induces differentiation into a somatic cell of interest.

As the cell to be cultured, a cell having a gene mutation or a cell derived from a patient may be used for the intended purpose of reproducing the pathological condition.

The cell culture base material may be used for the single culture of one kind of cell or for the co-culture of a plurality of kinds of cells. In a case of co-culturing a plurality of kinds of cells instead of simply culturing one kind of cell, cells may grow and proliferate in an environment more similar to the in vivo environment through intercellular interaction, which enhances biomimeticity.

The cell culture base material may be used for single faced culture or both faced culture. In a case of carrying out both faced culture, the kinds of cells cultured on each surface may be the same or different from each other. In particular, in a case where the porous membrane is a porous membrane having through-holes, cells on respective surfaces, during both faced culture, can interact well with each other through an extracellular matrix.

In one embodiment, a first cell may be cultured on one surface of the cell culture base material to form a first cell layer, and a second cell different from the first cell may be cultured on the opposite surface thereof to form a second cell layer.

More specifically, for example, by using a vascular endothelial cell layer as the first cell and by using a smooth muscle cell as the second cell, both kinds of the cells may be co-cultured, with the porous membrane being sandwiched, to produce a structure (a vascular wall model) mimicking the blood vessel. According to such a method, it is possible to improve the biomimeticity of the vascular wall model by the interaction between the vascular endothelial cell and the smooth muscle cell. Furthermore, since the cell culture base material has good cell adhesiveness, it is possible to produce a biological membrane having a small number of defects such as opening.

In the vascular wall model, it is preferable that chemical substances do not freely pass between the cells of the vascular endothelial cell layer, that is, it is preferable that a barrier function is provided. In the vascular wall model that is capable of being produced using the cell culture base material of the present disclosure, it is presumed that the intercellular adhesion of vascular endothelial cells develops in a state similar to the in vivo vascular wall. In order to carry out drug evaluation more accurately using vascular wall model, it is desirable that the vascular wall model has a structure and function similar to those of the in vivo vascular wall, and thus a vascular wall model that is capable of being produced by using the cell culture base material of the present disclosure can be an excellent tool for drug evaluation.

Cells may be seeded on the cell culture base material as a cell suspension by suspending the cells in a liquid medium. The liquid medium that is used for preparing a cell suspension or culturing cells is selected according to the kind of target cell. Specific examples of the medium include media optimized for cell types by adding a cell growth factor to a basal medium for mammalian cells, such as Dulbecco's Modified Eagle's Medium (DMEM), Dulbecco's Modified Eagle Medium:Nutrient Mixture F-12 (DMEM:F-12), Eagle's minimal essential medium (EMEM), Minimum Essential Medium Alpha (MEMa), and Basal Medium Eagle (BME).

Such media are available as commercially available products. The liquid medium may be a medium obtained by mixing a plurality of media. The pH of the liquid medium is, for example, pH 7.0 to 8.0.

«Cell Culture Base Material with Cells»

A cell culture base material with cells of the present disclosure has a cell layer on at least one surface of the above-described cell culture base material. The cell culture base material with cells can be obtained, for example, by seeding cells suspended in a liquid medium on the cell culture base material and culturing the cells. The above-described matters can be applied to the details of the cells in the cell layer and the cell culture base material.

### EXAMPLES

Hereinafter, embodiments of the present disclosure will be described in detail with reference to Examples. The embodiments of the present disclosure should not be interpreted restrictively by the following Examples.

### Example 1: Production of Cell Culture Base Material

The following porous membrane was used to produce a cell culture base material.
Honeycomb film made of polybutadiene (a porous membrane having a honeycomb structure, manufactured by FUJIFILM Corporation according to a known method such as the method disclosed in JP4945281B): It has an average opening diameter of 5 μm, a thickness of 1.7 μm, an opening ratio of 36%, a coefficient of variation of an opening diameter of 2%, and a pitch of 7.2 μm, where holes are through-holes, and adjacent holes are partitioned by a partition wall and connected by a communication hole.

The above honeycomb film was washed with ethanol and then immersed in a collagen I (rat tail, Corning Inc.) solution. The collagen solution was diluted with phosphate buffered saline (PBS) and sterile water to a concentration of 1 mg/mL and used. A 1 mol/L (N) sodium hydroxide aqueous solution was added so that the pH of the collagen solution was 8.5 and then mixed and ice-cooled. After immersing the honeycomb film in the ice-cooled collagen solution and then taking it out therefrom, the honeycomb film was allowed to stand at 37° C. for 30 minutes to gelate the collagen, whereby a cell culture base material (hereinafter, also referred to as HCF+Colgel) in which the holes of the honeycomb film were filled with the collagen gel was produced.

In addition, a honeycomb film not immersed in the collagen I solution (hereinafter, also referred to as an “untreated honeycomb film”) was prepared as a control.

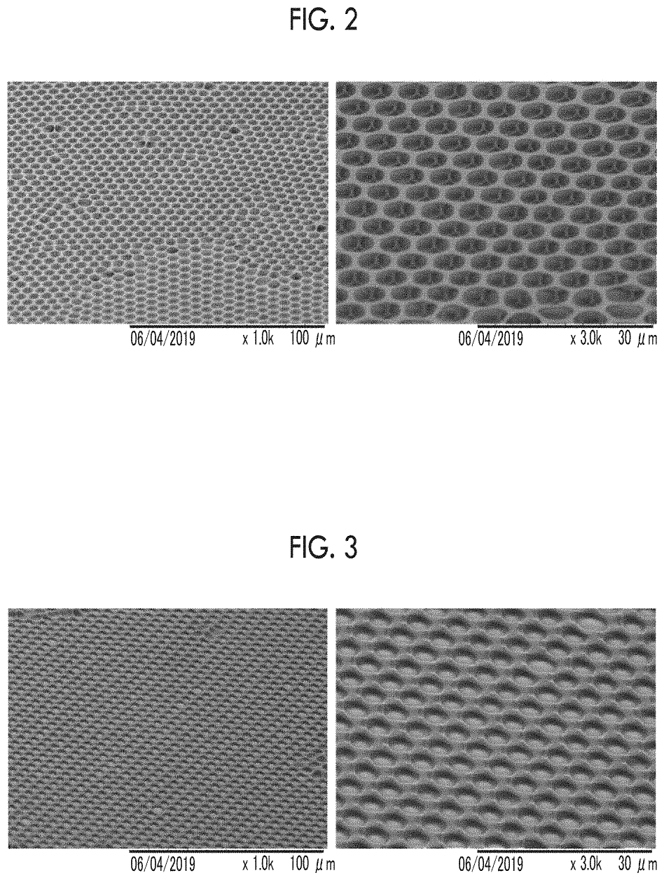

FIG. 2 shows observation photographic images of the untreated honeycomb film taken with a scanning electron microscope (SEM), and FIG. 3 shows observation photographic images of the cell culture base material (HCF+Colgel) produced as described above.

It is noted that as observed by SEM, the untreated honeycomb film shown in FIG. 2 and the HCF+Colgel shown in FIG. 3 are in a dry state; however, the cell culture base material shown in FIG. 3 is a planar cell culture base material in a moist state. It can be seen that the holes of the untreated honeycomb film shown in FIG. 2 are not filled with the collagen gel. It can be seen that the holes of the HCF+Colgel shown in FIG. 3 are filled with the collagen gel.

### Example 2: Cell Culture on Cell Culture Base Material

The following three kinds of cell culture base materials were prepared.

(Base material A) A cell culture base material (HCF) obtained by coating the honeycomb film with collagen I

The same honeycomb film as that used in Example 1 was immersed in a collagen I solution, subjected to a coating treatment with collagen I, and then washed with sterile water to produce a base material A.

It is noted that in the base material A, the surface of the honeycomb film is coated with collagen I; however, the collagen I is not gelated, and thus the holes are not filled with the collagen.

(Base material B) A cell culture base material (HCF+Colgel Low) obtained by filling the honeycomb film with a small amount of the collagen gel

A cell culture base material was produced by filling the holes of the honeycomb film with a collagen gel according to the method described in Example 1. The amount of collagen solution at the time of the immersion of the honeycomb film was set to a small amount so that the amount was an amount by which only the bottom part of the honeycomb film was immersed and thus only a part of the inside of the holes was filled with the collagen solution. The concentration of the collagen solution was set to 0.4 mg/mL.

The filling rate of the holes with the collagen gel was about 60%. Further, the thickness of the cell culture base material was 1.7

(Base material C) A cell culture base material (HCF+Colgel High) obtained by filling the honeycomb film with a large amount of the collagen gel

A cell culture base material was produced by filling the holes of the honeycomb film with a collagen gel according to the method described in Example 1. The amount of collagen solution at the time of the immersion of the honeycomb film was set to an amount so that the entire holes of the honeycomb film were filled with the collagen solution (that is, an amount by which the entire honeycomb film was immersed in the collagen solution).

The concentration of the collagen solution was set to 4.0 mg/mL. The hole filling rate with the collagen gel was about 100%. Further, the thickness of the cell culture base material was 1.7 μm.

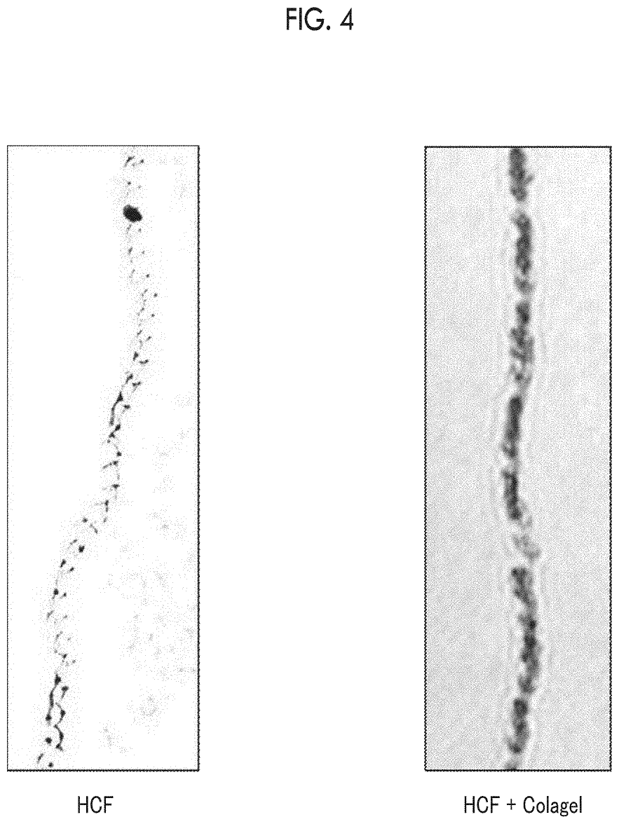

FIG. 4 shows cross-sectional images of the base material A and the base material C, observed with staining the collagen gel with a picrosirius red staining kit and observing with an optical microscope. The inside of the holes of the base material A shown in the left figure is not filled with the collagen gel; however, the inside of the holes of the base material C shown in the right figure is filled with the collagen gel.

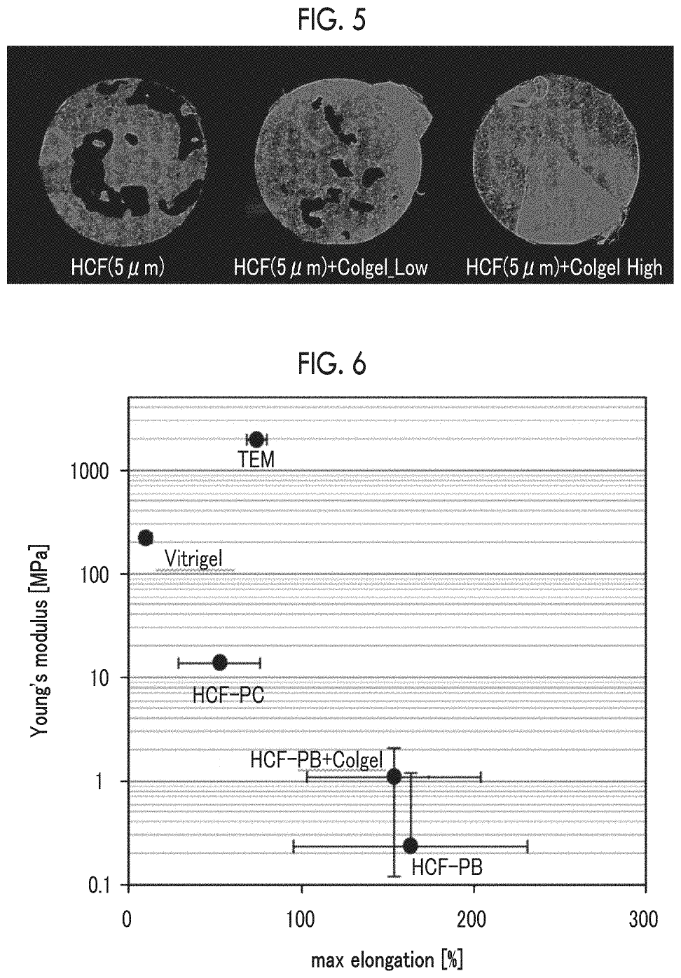

Rat vascular endothelial cell and smooth muscle cell were each seeded on one surface of the base materials A to C and co-cultured. After 8 days, the cultured cells were stained with VE-cadherin, and the culture surface was observed under a microscope. A microscopic image of each culture surface is shown in FIG. 5.

The proportion of the area occupied by the vascular endothelial cells covering the culture surface of the cell culture base material (hereinafter, also referred to as the coverage) was calculated according to the following expression. In the following expression, the area of the cell culture surface indicates the area of the portion of the cell culture base material in which cells have been seeded. That is, it can be said that the higher the coverage, the better the cell adhesiveness.

Coverage (%)={(area occupied by stained cultured cells)/(area of cell culture surface)}×100

The obtained coverage is shown below.

Base material A . . . 82.7±13.1%

Base material B . . . 90.4±0.4%

Base material C . . . 98.9±1.0%

As can be seen from the above results, the coverage in a case where cells were cultured using the base material B or the base material C was improved as compared with the coverage in a case where the cells were cultured using the base material A. In particular, it was confirmed that since the coverage in a case where the cells are cultured using the base material C is the highest, cell adhesiveness is also high, and that the higher the filling rate of the collagen gel in the holes of the honeycomb film is, the better the smooth muscle cells can be cultured.

### Example 3: Mechanical Properties of Cell Culture Base Material

The following five kinds of cell culture base materials were prepared.

(Base material D) A honeycomb film (HCF-PB) made of polybutadiene

The details thereof are the same as those of the honeycomb film used in Example 1, and the holes of the honeycomb film are not filled with the collagen gel.

(Base material E) A cell culture base material obtained by filling the holes of the honeycomb film made of polybutadiene with the collagen gel (collagen is in a moist state) (also referred to as HCF-PB+Collagen Gel (swelled) or HCF-PB+Colgel).

The production method is the same as that of the base material C of Example 2.

(Base material F) A track-etched membrane (TEM, manufactured by Merck KGaA) The opening ratio thereof is 20% or less.

(Base material G) A honeycomb film (HCF-PC) made of polycarbonate (produced by FUJIFILM Corporation according to a known method such as JP4945281B).

(Base material H) A collagen Vitrigel (collagen is in a moist state) (also referred to as Vitrigel (swelled) or Vitrigel, manufactured by Kanto Chemical Co., Inc.)

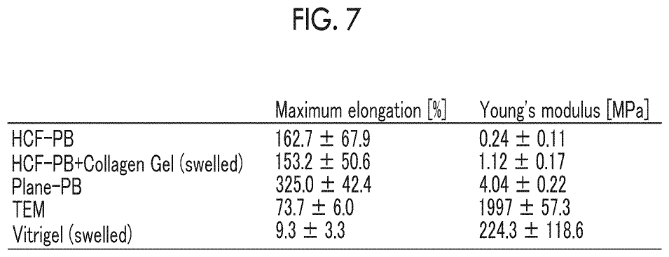

According to the following procedure, the base materials D to H were subjected to a tensile test based on JIS K 7161-1: 2014 and JIS K 7127: 1999, and the Young's modulus and the maximum elongation rate (max elongation or Maximum elongation) were determined. Specifically, samples cut out in a strip shape of 10 mm×30 mm were subjected to a tensile test using a force gauge manufactured by IMADA Co., Ltd. The Young's modulus was obtained from the slope of the elasticity region of the obtained stress-strain curve, and the maximum elongation rate was obtained from the strain at the time of rupture. All the tests were carried out three times, and the average value was calculated from the obtained values. The results are shown in FIG. 6 and FIG. 7.

As can be seen from FIG. 6 and FIG. 7, the base material E had a low Young's modulus and a high maximum elongation rate as compared with the base material F to the base material H. Further, the base material E had a Young's modulus and a maximum elongation rate comparable to those of the base material D. From the above results, it was found that the base material E has excellent deformability.

That is, the cell culture base material and the cell culture base material with cells according to the present disclosure, shown in Examples, are easily deformable and have excellent cell adhesiveness.

The disclosure of JP2019-194541 filed on Oct. 25, 2019, is incorporated in the present specification by reference in its entirety.

All of documents, patent applications, and technical standards described in the present specification are incorporated into the present specification by reference to approximately the same extent as a case where it is specifically and respectively described that the respective documents, patent applications, and technical standards are incorporated by reference.

What is claimed is:

1. A cell culture base material comprising:
a porous membrane having an opening ratio of 30% to 70%; and
an extracellular matrix with which an inside of a hole of the porous membrane is filled.

2. The cell culture base material according to claim 1,
wherein the porous membrane has an average opening diameter of 1 μm to 200 μm.

3. The cell culture base material according to claim 1,
wherein the cell culture base material has a thickness of 20 μm or less.

4. The cell culture base material according to claim 1,
wherein a filling rate of the hole with the extracellular matrix is 80% or more.

5. The cell culture base material according to claim 1,
wherein the extracellular matrix has a gel shape or is capable of forming a gel in a moist environment.

6. The cell culture base material according to claim 1,
wherein a Young's modulus, determined by a tensile test based on JIS K 7161-1: 2014 and JIS K 7127: 1999, is 2.0 MPa or less.

7. The cell culture base material according to claim 1,
wherein a maximum elongation rate, determined by a tensile test based on JIS K 7161-1: 2014 and JIS K 7127: 1999, is 150% or more.

8. The cell culture base material according to claim 1,
wherein at least one surface of the porous membrane is coated with an extracellular matrix.

9. A cell culture base material with cells, comprising a cell layer on at least one surface of the cell culture base material according to claim 1.
